(19) United States
(12) Patent Application Publication
(10) Pub. No.: US 2021/0178179 A1
(43) Pub. Date: Jun. 17, 2021

(54) DEVICE AND METHOD FOR STIMULATING PHOTO BIOCHEMICAL SYNTHESIS OF VITAMIN D3-CHOLECALCIFEROL

(71) Applicant: Andrey Smirnov, Moscow (RU)

(72) Inventor: Andrey Smirnov, Moscow (RU)

(21) Appl. No.: 17/113,443

(22) Filed: Dec. 7, 2020

(30) Foreign Application Priority Data
Dec. 11, 2019 (RU) 2019140870

(51) Int. Cl. A61N 5/06

(52) U.S. Cl. CPC A61N 5/0613; A61N 2005/0645; A61N 2005/0626; A61N 2005/0661

(57) ABSTRACT

Disclosed herein are devices for stimulating the photo biochemical synthesis of vitamin D3-cholecalciferol in the body. The device includes a housing (1), a power section (2) located in the housing, a section (3) for parameter-setting and for narrowband UVB radiation regulation, a screen or touch screen (4) for visualization, a source (5) for narrow-band UVB radiation, and a module (6) for uniform distribution of the narrow-band UVB radiation, which can be in direct contact with the skin. The technical result of the claimed invention is to provide stimulation of the synthesis of the required daily dose of vitamin D3-cholecalciferol using low intensity narrow-band radiation in the UVB range, which makes it possible to increase the concentration of vitamin D3-cholecalciferol to the required daily norm.

### CROSS-REFERENCE TO RELATED APPLICATIONS

This application claims priority to Russian Patent Application No. 2019140870, filed Dec. 11, 2019, now Russian Patent No. 2730551, which is hereby fully incorporated by reference herein.

### BACKGROUND

The invention relates to medical technology, in particular to devices for stimulating the photo biochemical synthesis of vitamin D3-cholecalciferol.

The energy of solar radiation when exposed to the skin is converted into heat and is also spent on photochemical and photobiological reactions, during which various photo biochemical processes occur in the skin's epidermis. In 1935, the chemical structure of vitamin D3 was established, and it was proved that vitamin D3 is formed as a result of ultraviolet irradiation of 7-dehydrocholesterol. Depending on the dose and quality of UV radiation, the physiological effect can be either positive (vitamin D3 synthesis) or negative (burn, DNA damage, the appearance of malignant formations on the skin, photoaging, etc.). The human body is supplied with vitamin D in two ways: (1) endogenous formation of vitamin D3 in the epidermis of the skin when it is exposed to UV radiation of a certain spectrum, and (2) by the absorption of exogenous vitamin D2, D3 in the intestine from the food. With sufficient and regular insolation, the need for vitamin D is fully covered by the photochemical synthesis in the epidermis. Dietary sources of vitamin D2, D3 only serve as a compensating role in cases of endogenous vitamin deficiency. The activity of photo biochemical stages of vitamin D3 synthesis is directly dependent on the intensity and spectral composition of UV radiation (UVR). The photo biochemical process of vitamin D3 synthesis occurs only when the skin is exposed to the energy of ultraviolet radiation with certain characteristics. 7-Dehydrocholesterol most effectively absorbs ultraviolet radiation in the UVB range (280-315 nm). Thus, vitamin D3 production occurs most efficiently at these wavelengths.

The amount of UV radiation reaching the skin is extremely important for the entire vitamin D3 synthesis system. The required dosage is small, and daily exposure of the face and hands to the sun and light for 15 minutes is considered sufficient. It is noteworthy that exposure to higher doses of radiation in the UVB range (280-315 nm) on larger areas of the body gives a less favorable ratio of risk and benefit from UVR. Studies have revealed a state of saturation in the production of vitamin D with an increase in the body area. The benefit/risk ratio appears to be favorable only for small doses of UVB radiation and small areas of the body, and excessive exposure to the sun does not provide proportionally additional benefits. The synthesis of vitamin D3 in the skin occurs under UV irradiation, which effectively penetrates only the epidermal layer of the skin. The two most important factors that affect vitamin D3 synthesis are the amount (intensity) and quality (corresponding wavelengths) of UV radiation. Another important factor is the amount of 7-dehydrocholesterol in the skin. Under normal conditions, the papillary and basal layers of the skin contain a sufficient amount of 7-dehydrocholesterol (about 25-50 mcg/cm² of the skin). In vitro skin samples showed that the amount of vitamin D3 reaches its maximum within 15 minutes after UV irradiation, and during prolonged exposure its amount remains constant. Thus, to fully meet the daily needs of the body through endogenous vitamin D3, a 10-20-minute stay in open sunlight containing UVB rays is sufficient.

In conditions of sunlight deficiency, vitamin D deficiency can be compensated by stimulating photo biochemical synthesis of vitamin D3 using a vitamin D3-cholecalciferol synthesis device in the body by irradiating the skin with narrow-band radiation in the UVB range.

There are devices disclosed in U.S. Pat. No. 6,851,814 and Russian Patent No. RU2644752, in which the synthesis of vitamin D in the body is carried out by irradiating the skin using UVB lamp radiation sources that emit at wavelengths close to the biologically active radiation spectrum for the production of vitamin D.

The closest in terms of a set of essential features is a device according to Korean Patent Application KR20150082761 which contains a light-emitting diode, a light guide, a control central processor, a scattering plate for uniform distribution of radiation over the surface of the device, all assembled on a flexible printed circuit board, and a power supply. In such a device, the required dose of vitamin D is achieved with high-intensity UVB radiation with a wavelength of 280 nm, which requires, to ensure the safety of the irradiation process, setting and monitoring the maximum permissible exposure time (work shift, sleep time, etc.) and the safe lowest dose of narrow-band UVB radiation corresponding to a long exposure time. This drawback is also typical for technical solutions under the above-mentioned U.S. Pat. No. 6,851,814 and Russian Patent No. RU2644752.

### BRIEF DESCRIPTION

The object of the present invention is to create a device that provides narrow-band radiation in the UVB range for energy-efficient and safe stimulation of the synthesis of vitamin D3-cholecalciferol in the body.

The technical result of the claimed invention is to provide stimulation of the synthesis of the required daily dose of vitamin D3-cholecalciferol at a low intensity of narrow-band radiation in the UVB range, which makes it possible to increase the concentration of vitamin D3-cholecalciferol to the required daily norm.

This technical result is achieved by a method and device for stimulating the synthesis of vitamin D3 cholecalciferol in the body UVB radiation, comprising: (A) a housing, and located in the housing, (i) a power section, (ii) a section for parameter-setting and for regulation of the narrow-band UVB radiation, (iii) a narrow band UVB source of radiation and (iv) a module for uniform distribution of the narrow-band UVB radiation onto an irradiated surface, which is located in the lower part of the housing opposite the source of narrowband UVB radiation.

A screen, which may be a touch screen, can be used for visualization and input and is placed on the upper surface of the housing. Buttons may also be present for input.

The aforementioned section (ii) for parameter-setting and for regulation of the narrow-band UVB radiation contains (a) a regulation module for regulating the electrical parameters of input voltage and current, (b) a regulation module for regulating the power control of the narrow-band UVB radiation, and (c) a module for setting parameters and adjusting the power of the narrow-band UVB radiation, the exposure modes, and control (on/off) source of the UVB radiation, or in other words a control module for the user to determine how the device operates.

The module (iv) for uniform distribution of narrow-band UVB radiation can be a plate made of plastic or other material that is transparent to UVB radiation, isotropic through its entire volume, and that scatters UVB radiation from the source/sources of UVB radiation over the irradiated surface.

In other preferred embodiments, the plate in the module (iv) for uniform distribution of narrow-band UVB radiation is made of a UVB transparent isotropic plastic material. The plate should have a thickness of about 0.5 millimeters (mm) to about 3 mm. The plastic should be isotropic through its entire volume with a transmittance of at least 50% of UVB radiation. In some versions, the plate can be formed from a suspension of colloidal silicon particles and anti-sedimentation additives that additionally stabilize the uniform distribution of fillers and reduce the formation of large agglomerations of particles. The suspension can then be polymerized in a mold to form a plate having a desired shape, thickness, and volume. The plate can alternatively be an optical system that evenly scatters UVB radiation over the irradiated surface, such as a scattering lens, a Fresnel lens, or a collimator. Due to the isotropic property of the material, a uniform distribution of narrow-band radiation from a UVB diode or a group of UVB diodes radiating in the UVB range occurs over the entire surface of the plate, and accordingly over the surface of the skin which the module contacts. The use of such a narrow-band UVB radiation and distribution module makes it possible to perform irradiation at a minimum UVB radiation power.

In desirable embodiments, the module (iv) for uniform distribution of narrow-band UVB radiation should be in direct contact with the skin of the human user.

To ensure safe radiation conditions during the synthesis of vitamin D3 in the body by narrow-band UVB radiation, a narrow-band led UVB radiation source with a wavelength in the range of 290-301 nanometers (nm) with a main wavelength of 298 nm is used as a source of narrow-band UVB radiation to create a daily radiation dose of 0.15 MED.

In some preferred embodiments, a narrow-band light-emitting diode (LED) with a radiation emission range of 290-301 nm with a main radiation wavelength of 298 nm is used as the source of narrow-band UVB radiation.

The device according to the invention can be part of a bracelet, as well as a bracelet attached to the body, in which the module (iv) for uniform distribution of narrow-band UVB radiation is in direct contact with the skin of the body.

These and other non-limiting characteristics of the disclosure are more particularly disclosed below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a block diagram of a device for the synthesis of vitamin D3-cholecalciferol according to the invention.

FIG. 2 is a block diagram of a narrow-band UVB radiation generation unit for the synthesis of vitamin D3-cholecalciferol according to the invention.

FIG. 3 is a graph showing the relative spectral distribution of a 295 nm UVB LED diode suitable for use in the device of the present disclosure. This diode has a range of approximately 280 nm to 330 nm.

FIG. 4 is a graph showing the relative spectral distribution of a 290 nm UVB LED diode suitable for use in the device of the present disclosure. This diode has a range of approximately 275 nm to 310 nm.

FIG. 5 is a graph showing the relative spectral distribution of a 300 nm UVB LED diode suitable for use in the device of the present disclosure. This diode has a range of approximately 280 nm to 330 nm.

FIG. 6 is an illustration of a bracelet on the wrist of a user.

### DETAILED DESCRIPTION

A more complete understanding of the components, processes and devices disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the terms “comprise(s),” “include(s),” “having,” “has,” “can,” “contain(s),” and variants thereof, as used herein, are intended to be open-ended transitional phrases, terms, or words that require the presence of the named components/steps and permit the presence of other components/steps. However, such description should be construed as also describing systems or devices or compositions or processes as “consisting of” and “consisting essentially of” the enumerated components/steps, which allows the presence of only the named components/steps, along with any unavoidable impurities that might result therefrom, and excludes other components/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

A value modified by a term or terms, such as “about” and “substantially,” may not be limited to the precise value specified. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number.

The term “narrow-band” as used herein refers to a wavelength range wherein all spectral components of the radiation are grouped within ±30 nm of the central wavelength.

The light transmittance refers to the ratio of transmitted light to incident light, is reported in percentages, and may be measured according to ASTM D1003-13.

The term “UVB” as used herein refers to radiation in the range from 280 nanometers to 315 nanometers (two hundred eighty to three hundred fifteen nm). When referring to UVB radiation in this application, only the central wavelength must be within this range.

The term “MED” as used herein refers to Minimum Erythema Dose. Erythema itself is a limited intense redness of the skin caused by the dilation of the vessels of the dermis. The MED corresponds to an energy exposure of about 150 J/m² to about 2000 J/m², depending on skin type, at a wavelength of 298 nm (the maximum spectral efficient wavelength for erythema).

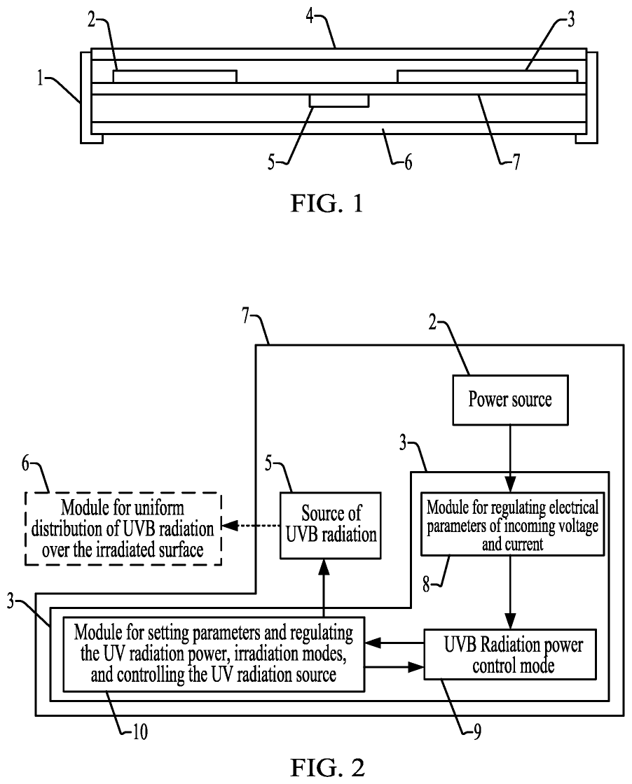

FIG. 1 shows a block diagram of the device for the synthesis of vitamin D3-cholecalciferol according to the invention. The device comprises a housing 1, which contains a section 2 for power supply, a section 3 for parameter-setting and narrow-band UVB radiation regulation, a screen or touch screen 4 for visualization and device mode input, a source 5 for narrow-band UVB radiation, and a module 6 for the uniform distribution of narrow-band UVB radiation on the irradiated surface. The section 2 for power supply, the section 3 for parameter-setting and narrow-band UVB radiation regulation, and the source 5 for narrow-band UVB radiation are located on circuit board 7.

As a source of narrow-band UVB radiation, a narrow-band light-emitting diode (LED) radiation source in the UVB range can be used: one narrow-band UVB diode can be used, or several narrow-band UVB diodes can be evenly placed opposite the module for uniform distribution of narrow-band UVB radiation. The radiation dose provided by a narrow-band UVB diode source/sources is assumed to be 0.15 MED/day. If a single source of narrow-band UVB radiation is used, the daily radiation dose (energy) should be 0.15 MED or the combined dose of narrow-band UVB radiation sources should be 0.15 MED. It is noted that typically, such sources of narrow-band UVB radiation do not also provide significant amounts of radiation outside of the narrow band.

FIG. 2 shows a block diagram of the UVB radiation generation unit. The power source 2 provides power to the components of the device that generates UVB radiation with the required parameters. As a power source, a battery installed in the compartment of the power supply can be used. To charge the battery, the power supply compartment can be equipped with a charging port (for example a mini USB port) or charging can occur from a DC source.

Section 3 for parameter-setting and narrowband UVB radiation regulation provides the power regulation of the narrow-band UVB radiation and contains module 9 to regulate the output of narrow-band UVB radiation and module 10 that sets the parameters and adjusts the power of the narrow-band UVB radiation, modes of exposure, and source control narrow-band UVB radiation. This module 10 includes a software node for setting device operation parameters and can be made, among other things, as a controller (for example, a chip) regulating the current strength, or PWM (Pulse Width Modulation), i.e. radiation power. The power supply of the mentioned modules 9 and 10 is provided through module 8 for regulating the electrical parameters of the incoming voltage and current. Module 8 may be, for example, an input voltage converter.

Module 10 receives the data of performance parameters of irradiation specified by the module 9 of narrow-band UVB radiation power control, that are set on the screen or touch screen, and through an integrated software module generates an output signal supplied to the source 5 of narrow-band UVB radiation. The module also provides a mode for switching on/off the narrow-band UVB radiation source 5 in accordance with the specified irradiation period, which is provided by the communication of module 10 with the on/off tool of the source 5 of narrow-band UVB radiation. The UVB radiation power is regulated by current regulation or PWM and is set via the device's screen interface. The radiation power is adjusted when it is necessary to change the irradiation time (increase/decrease). For example, current control by module 10 permits control of the radiometric radiation power of the UVB source 5. Regulation of the current strength can occur at a step of 1 mA using the process of pulse-width modulation. This process does not cause changes in other electrical parameters. The charging value can be shown on the screen interface 4 as a percentage of the battery capacity. The value of the radiometric radiation power can also be controlled/changed by (+) and (−) buttons (either physical buttons or virtual buttons on a touch screen) that decrease or increase the radiometric radiation power in percent with a step of 1%. The required radiation power can be based on the value of 0.15 MED, the required minimum value of UVB radiation to obtain a daily increase in vitamin D3 of endogenous origin.

The mentioned module 8, module 9, and module 10 can be placed on the same printed circuit board or integrated into a single chip. Modules 9 and 10 may be implemented on one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. These modules are capable of being implemented as hardware, software, or any suitable combination thereof. In general, any device capable of implementing a finite state machine can be used for these modules.

The module 6 for the uniform distribution of narrow-band UVB radiation can be made from a plate or an optical system. The plate can have a thickness of about 0.5 mm to about 3 mm. The plate can be made, for example, from a plastic material that is transparent to UVB radiation, and isotropic in its entire volume with a transmission coefficient of at least 50%. The plate can be made, for example, from a suspension of colloidal silicon particles (i.e. up to about 100 nm in diameter) and anti-sedimentation additives that additionally stabilize the uniform distribution of fillers and reduce the formation of large agglomerations of particles. The suspension can then be polymerized in a mold to form a plate having a desired shape, thickness, and volume. All components are evenly distributed throughout the volume. Alternatively, an optical system is used to evenly scatter UVB radiation over the irradiated surface against which the module 6 is placed. Such optical systems can include a scattering lens, a Fresnel lens, a collimator, etc. Due to the isotropic property of the material used in module 6, a uniform distribution of narrow-band radiation is provided from a UVB light-emitting diode or a group of UVB light-emitting diodes radiating in the UVB range (which can be used as radiation source 5) over the entire external-facing surface of the module and, accordingly, over the surface of the skin with which the module is in contact with. The use of such a narrow-band UVB radiation distribution module makes it possible to perform irradiation at the minimum required UVB radiation power. An example of a suitable material for the plastic material is SILGEL 612, available from Wacker Silicones.

Power from the power source 2 is supplied to the module 8 for regulating electrical parameters of incoming voltage and current and further to the module 9 for narrow-band UVB radiation power control and to the module 10 for setting parameters and regulating the UV radiation power, irradiation modes, and controlling the UV radiation source.

The housing is made, for example, of durable plastic or metal and defines the radiation zone, limited by the size of the housing itself, or the housing and the bracelet at the same time, while the radiation zone is made with the possibility of placing a part of the body under it. The irradiation zone is preferably an area of about 8 to about 30 sq cm (cm²), including from about 10 to about 20 sq cm (cm²), or an even greater area, based on the fact that 1 cm² contains about 25-50 micrograms (mcg) of 7-dehydrocholesterol. Put another way, the surface area of the module 6 from which the UVB radiation escapes the device is about 8 cm² to about 30 cm², or from about 10 cm² to about 20 cm².

As previously mentioned, the device includes a screen or touch screen. The screen or touch screen 4 receives feedback from the module 9 for narrow-band UVB radiation power control and the power source 2 to monitor the operating parameters. Known input means, such as buttons or dials or the touch screen itself, can be used to set various parameters as desired.

The device according to the invention can also be equipped with a receiver for remote control signals for setting radiation mode parameters associated with the software being operated by module 10 for setting radiation parameters.

The device and method described above for stimulating the synthesis of vitamin D3-cholecalciferol in the human body can be used to irradiate various parts of the body. In a preferred embodiment, such a device may be part of a bracelet or the entire bracelet that is attached to the arm or leg. The bracelet is in the shape of a ring, having an upper surface and a lower surface that contacts the skin of the human user. In this case, the module 6 for the uniform distribution of narrow-band UVB radiation is in direct contact with the skin of the body.

Configuring the device consists of setting a regular dose of radiation depending on the skin type. Based on data from studies of ultraviolet radiation, a daily recommended radiation dose energy is 0.15 MED. Depending on the irradiation power, the exposure time (irradiation) is set. On the screen of a device, for example, integrated with a bracelet, the exposure time will be set. When choosing a different exposure time, the irradiation power will change proportionally (by reducing/increasing the current or PWM). For other skin types with a high content of melanin, the radiation power may increase. This device according to the invention is intended for all skin types according to the Fitzpatrick Scale. Irradiation is carried out at wavelengths of 290-301 nm with the main wavelength of 298 nm. At these wavelengths, the most effective process of vitamin D3 synthesis in the body is provided—up to 90% relative to 298 nm with a low level of negative effects of radiation, in particular, erythema, which is confirmed by the data of the ICO (International Commission on Lighting) shown in the table below. At the same time, the direct contact of the module 6 for uniform distribution of radiation practically eliminates its impact on the people around the user. The table shows in bold the wavelength of 298 nm of the radiation spectrum, which allows to get the best result of vitamin D3 synthesis.

TABLE
erythema level and vitamin D synthesis as a function of
wavelength (CIE 174: 2006 Action Spectrum for the
production of provitamin D3 in Human Skin Standard by
Commission Internationale de l'Eclairage, Jan. 1, 2006).

| No. | Wavelength, nm | Erythema (%) | Vitamin-D (%) |
|---|---|---|---|
| 1 | 290.90 | 1.0000000 | 0.9005000 |
| 2 | 292.00 | 1.0000000 | 0.9280000 |
| 3 | 293.00 | 1.0000000 | 0.9520000 |
| 4 | 294.00 | 1.0000000 | 0.9760000 |
| 5 | 295.00 | 1.0000000 | 0.9830000 |
| 6 | 296.00 | 1.0000000 | 0.9900000 |
| 7 | 297.00 | 1.0000000 | 0.9960000 |
| 8 | 298.00 | 1.0000000 | 1.0000000 |
| 9 | 299.00 | 0.8053780 | 0.9770000 |
| 10 | 300.00 | 0.6486340 | 0.9510000 |
| 11 | 301.00 | 0.5223960 | 0.9170000 |
| 12 | 301.40 | 0.4790720 | 0.9014000 |

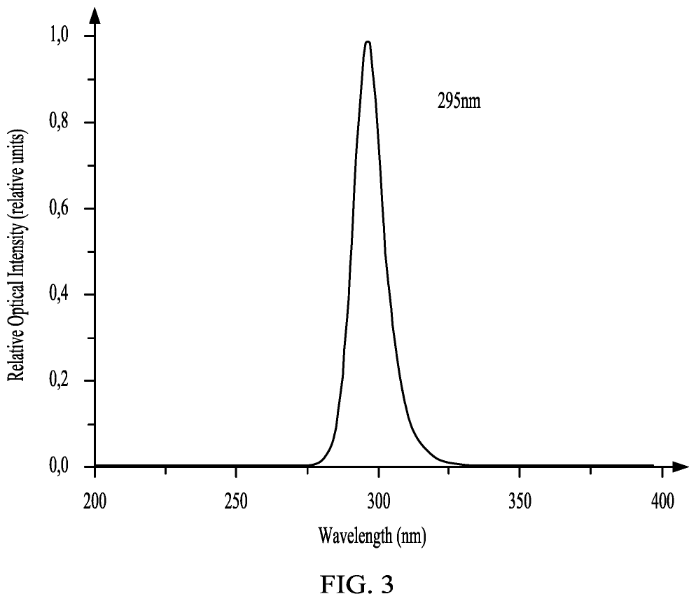

The device was tested in in vivo experiments. For skin irradiation, a UVB light-emitting diode from Dongguan Houke Electronic Co., Ltd was used. The peak wavelength of the LED was 295 nm, and its spectrum is shown in FIG. 3.

The measurements were performed in a medical laboratory by passing a blood test for 25(OH)D3 concentration before and after irradiation, 24 hours after the end of the irradiation session. The following results were obtained:

1. Results of the first study (Option 1) on radiation exposure using a wristband device. Irradiation took place for 5 days in conditions of insufficient sunlight. Sessions were performed once a day with an exposure time of 900 seconds. After the end of the irradiation, there was no radiation for the next 24 hours to complete the production of vitamin D3-cholecalciferol. The energy of the biologically active radiation spectrum transmitted through the module for uniform distribution of radiation to the human skin was 0.005 J/cm² (5 mJ/cm²), taking into account the loss of UVB radiation passing through the scattering layer—about 20%. After 24 hours, the concentration of 25-hydroxycholecalciferol (25(OH)D3) in the blood increased from 12.82 ng/ml to 17.52 ng/ml, which was 4.7 ng/ml, or 32.9 micrograms, or 1293 IU for 5 days of irradiation using a device for stimulating the photo biochemical synthesis of vitamin D3-cholecalciferol, using narrow-band UVB irradiation. The daily increase in the concentration of 25-hydroxycholecalciferol in the blood was 6.58 mcg or 263.2 IU.

2. Results of the second study (Option 2) on radiation exposure using a device in the form of a wristband. Irradiation occurred for 6 days in conditions of insufficient sunlight. Sessions were performed once a day with an exposure time of 900 seconds. After the end of the irradiation, there was no radiation for the next 24 hours to complete the production of vitamin D3-cholecalciferol. The energy of the biologically active radiation spectrum transmitted through the module of uniform distribution of radiation to the human skin was 0.005 J/cm² (5 mJ/cm²), taking into account the loss of UVB radiation passing through the scattering layer—about 20%. After 24 hours, there was an increase in the blood concentration of 25-hydroxycholecalciferol from 23.79 ng/ml to 33.17 ng/ml, which was 9.38 ng/ml, or 65.66 mcg, or 2626.4 IU for 6 days of irradiation using a device for stimulating photo biochemical synthesis of vitamin D3, narrow-band UVB irradiation. The daily increase in the concentration of 25-hydroxycholecalciferol in the blood was 10.943 mcg or 437.73 IU.

The examples of the implementation of the invention show the possibility of obtaining a technical result in all the wavelength intervals listed in the table above.

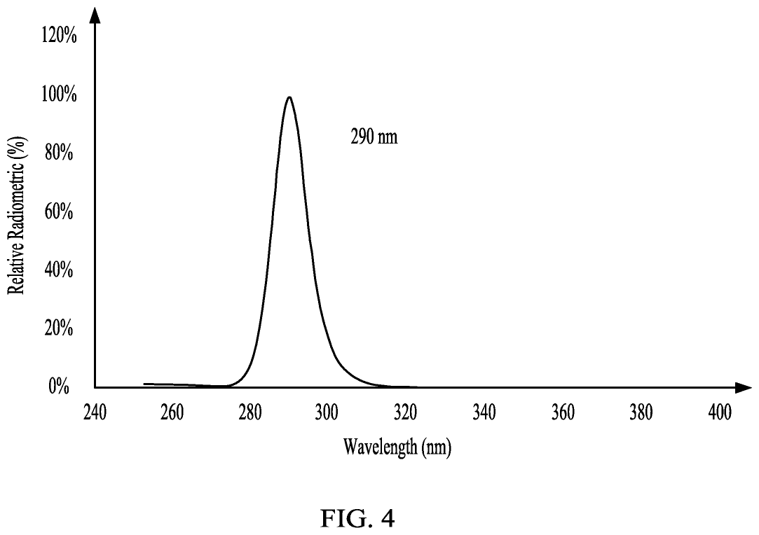

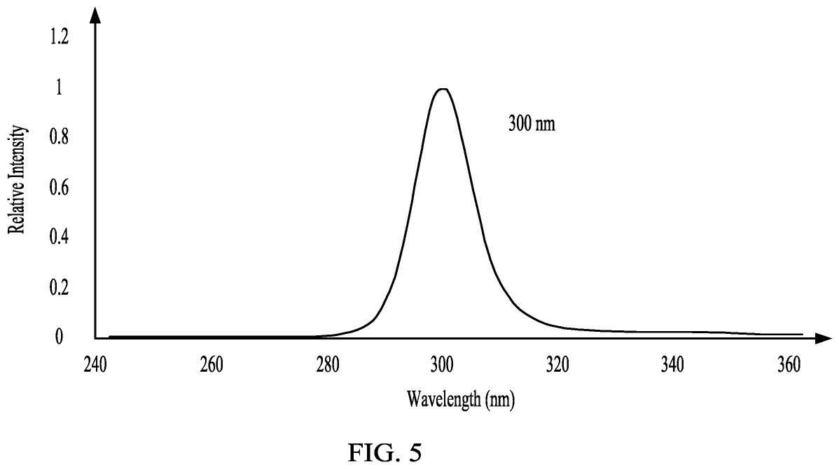

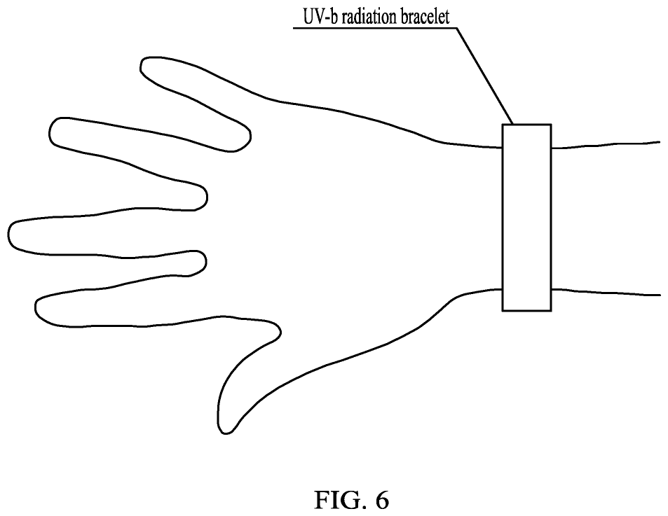

Additional in vivo experiments were performed using UVB diodes at 290 and 300 nm wavelengths. A UVB light-emitting diode from Dongguan Houke Electronic Co., Ltd was used for skin irradiation in each experiment. The peak wavelength of the LEDs was 290 nm and 300 nm, and their spectrum is shown in FIG. 4 and FIG. 5, respectively. The measurements were performed in a medical laboratory by passing a blood test for 25(OH)D3 concentration before and after irradiation, 24 hours after the end of the irradiation session.

3. UVB diode emitting at a wavelength of 290 nm (Option 3). Results of a study on radiation exposure using a device in the form of a bracelet on the wrist. Irradiation took place for 2 days in conditions of insufficient sunlight. The session was performed once a day with an exposure time of 900 seconds. After the end of the irradiation, there was no radiation for the next 24 hours to complete the production of vitamin D3-cholecalciferol. The energy of a regulated narrow-band UVB radiation source at 290 nm was 0.005 J/cm², the irradiation area was 20 cm². When passing through the module of uniform distribution of narrow-band UVB radiation, there is a loss of about 20% of energy for scattering. After 24 hours, there was an increase in the concentration of 25-hydroxycholecalciferol in the blood from 21.27 ng/ml to 23.75 ng/ml, which was 2.48 ng/ml, or 17.36 mcg, or 694.4 IU for 2 days of irradiation using a device for stimulating the photo biochemical synthesis of vitamin D3-cholecalciferol, narrow-band UVB irradiation. The daily increase in the concentration of 25-hydroxycholecalciferol in the blood was 8.68 mcg or 347.2 IU.

4. UVB diode emitting at a wavelength of 300 nm (Option 4). Results of a study on radiation exposure using a device in the form of a bracelet on the wrist. Irradiation took place for 2 days in conditions of insufficient sunlight. The session was performed once a day with an exposure time of 900 seconds. After the end of the irradiation, there was no radiation for the next two days to complete the production of vitamin D3-cholecalciferol. The energy of a regulated narrow-band UVB radiation source at 300 nm was 0.005 J/cm², the irradiation area was 20 cm². When passing through the module for uniform distribution of narrow-band UVB radiation, there is a loss of about 20% of energy for scattering. After 24 hours, there was an increase in the concentration of 25-hydroxycholecalciferol in the blood from 23.41 ng/ml to 26.18 ng/ml, which was 2.77 ng/ml, or 19.39 mcg, or 775.6 IU for 2 days of irradiation using a device for stimulating the photo biochemical synthesis of vitamin D3-cholecalciferol, narrow-band UVB irradiation. The daily increase in the concentration of 25-hydroxycholecalciferol in the blood was 9.695 mcg or 387.8 IU.

These studies are shown in Table 1:

TABLE 1

| | | | | | Increase in |
|---|---|---|---|---|---|
| | | | Energy of | | concentration |
| | | | irradiation of | | 25 (OH)D3 |
| | | | an area of | Number of | in blood |
| Wavelength | Voltage | Current | 20 sq. cm | Days of | (mcg) in |
| (nm) | (V) | (mA) | (mJ) | Irradiation | 24 hours |
| 290 | 7 | 40 | 100 | 2 | 8.68 |
| 295 | 6.5 | 40 | 100 | 5 | 6.58 |
| 295 | 6.5 | 40 | 100 | 6 | 10.943 |
| 300 | 7 | 40 | 100 | 2 | 9.695 |

The accepted thickness range of about 0.5 mm to about 3 mm of the device with a transmittance of at least 50% for a plastic plate was obtained experimentally. In this range, acceptable indicators of vitamin D3 synthesis were obtained when narrow-band radiation passed through the plate in the wavelength range of 290-301 nm and at controlled radiation energy of 0.005 J/cm² (5 mJ/cm²), which provides the lowest intensity of narrow-band UVB radiation. When using a plate with the specified thickness range, a minimum daily dose of 0.15 MED was provided for the entire wavelength range of UVB radiation from the LED. With these plate thicknesses, the bracelet-shaped device has a case size in height that does not exceed the size of wearable electronic devices, such as smartwatches or devices for health monitoring.

The present invention is not limited to the above versions of the device for the synthesis of vitamin D3 in the body.

For example, it is possible to simultaneously use a UVB radiation source and a diode IR radiation source. With simultaneous ultraviolet and infrared radiation, short-wave radiation is superimposed on long-wave radiation. The infrared flux modulated by ultraviolet radiation is a carrier of ultraviolet energy, providing its deeper penetration into the subcutaneous layer, ensuring its energetic interaction with biochemical processes, for example, the synthesis of vitamin D3.

The combination of the isotropic property of the material of the module 6 of the device according to the present invention and the radiation range of the UVB diode from 290 to 301 nm with the main wavelength of 298 nm ensures the operation of the device at a low intensity of UVB radiation, which makes it possible to increase its energy efficiency and safety for the user and people around.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A device for stimulating the photo biochemical synthesis of vitamin D3-cholecalciferol, comprising:
a housing (1), and located within the housing:
a power section (2);
a narrow-band UVB radiation source (5);
a section for controlling the narrow-band UVB radiation (3); and
a module (6) for uniform distribution of the narrow-band UVB radiation, located at a bottom surface of the housing (1);
wherein the section for controlling the narrow-band UVB radiation (3) contains:
a module (8) for regulating incoming voltage and current;
a module (9) for controlling the narrow-band UVB radiation power; and
a module (10) for setting parameters for UVB radiation exposure.

2. The device according to claim 1, wherein the module (6) for uniform distribution of the narrow-band UVB radiation includes a plate that is transparent to UVB radiation and is isotropic throughout its volume, and is configured to be in direct contact with the skin of a user.

3. The device according to claim 2, wherein the plate transmits at least 50% of the narrow-band UVB radiation.

4. The device according to claim 2, wherein the plate is formed from a suspension of colloidal silicon particles and anti-sedimentation additives, which is polymerized in a given volume.

5. The device according to claim 2, wherein the plate has a thickness of about 0.5 mm to about 3 mm.

6. The device according to claim 1, wherein the module (6) for uniform distribution of the narrow-band UVB radiation is in the form of an optical system that evenly scatters UVB radiation over an irradiated surface.

7. The device according to claim 1, wherein the module (6) has a radiation-emitting area of about 8 cm² to about 30 cm².

8. The device according to claim 1, wherein the narrow-band UVB radiation source (5) is an LED with a main wavelength of 298 (−8/+3) nm.

9. The device according to claim 1, wherein the narrow-band UVB radiation source (5) is a plurality of LEDs, each LED having a main wavelength of 298 (−8/+3) nm.

10. The device according to claim 1, wherein the power section (2), the narrow-band UVB radiation source (5), and the section for controlling the narrow-band UVB radiation (3) are on a circuit board (7).

11. The device according to claim 1, further comprising a screen or touch screen (4) on an upper surface of the housing.

12. The device according to claim 1, wherein the device is in the form of a bracelet.

13. A method for stimulating the photo biochemical synthesis of vitamin D3-cholecalciferol, comprising:
donning a device according to claim 1; and
activating the device to produce narrow-band UVB radiation which stimulates the synthesis of vitamin D3-cholecalciferol.

14. The method according to claim 13, wherein the device provides a daily narrow-band UVB radiation dose of 0.15 MED.
